(19) United States
(12) Patent Application Publication
(10) Pub. No.: US 2012/0149064 A1
(43) Pub. Date: Jun. 14, 2012

(54) Filamentous Fungi With Impaired PTRB Activity For Altered Protein Production

(75) Inventors: Huaming Wang, Fremont, CA (US); Guomin Tang, Beijing (CN); Aoquan Wang, Beijing (CN); Jinxiang Zhang, Beijing (CN)

(73) Assignee: Danisco Us Inc., Palo Alto, CA (US)

(21) Appl. No.: 13/129,994

(22) PCT Filed: Nov. 17, 2009
(86) PCT No.: PCT/US09/64790

§ 371 Date: Jul. 28, 2011

Related U.S. Application Data
(60) Provisional application No. 61/115,818, filed on Nov. 18, 2008.

(51) Int. Cl. C12P 21/00; C12N 1/15

(52) U.S. Cl. 435/71.1; 435/254.11; 435/254.3; 435/254.6; 435/254.8; 435/254.9

(57) ABSTRACT

A filamentous fungal cell is provided comprising at least one mutation, wherein the filamentous fungal cell has impaired ptrB activity and has altered expression of a protein of interest as compared to a corresponding parent filamentous fungal cell. In one embodiment, the altered expression of the protein of interest is enhanced expression of the protein of interest.

### PRIORITY

The present application claims priority to U.S. Provisional Application Ser. No. 61/115,818, filed on Nov. 18, 2008, which is hereby incorporated by reference.

### BACKGROUND

Genetic engineering has allowed improvements in microorganisms used as industrial bioreactors, cell factories and in food fermentations. Important enzymes and proteins produced by engineered microorganisms include glucoamylases, a-amylases, cellulases, neutral proteases, and alkaline (or serine) proteases, hormones and antibodies. However, the occurrence of protein degradation and modification in some genetically engineered systems can interfere with efficient production.

Filamentous fungi (e.g., Aspergillus and Trichoderma species) and certain bacteria (e.g., Bacillus species) have been engineered to produce and secrete a large number of useful proteins and metabolites (see e.g., Bio/Technol. 1987. 5: 369-376, 713-719 and 1301 -1304 and Zukowski, “Production of commercially valuable products,” In: Doi and McGlouglin (eds.) Biology of Bacilli: Applications to Industry,© 1992, Butterworth-Heinemann, Stoneham. Mass pp

Improvement of heterologous protein production has been one of main focused research areas in Aspergillus niger. The low yield is caused primarily by proteolytic degradation and slow protein folding process during the secretion. Several extracellular proteases have been studied in A. niger. Mutant strains lacking these proteases were also isolated and strains have been used to improve production of the heterologous proteins. Deletion of three aspartic proteases showed heterologous laccase production improvement 5 to 37% (Wang, Y., et al., Fungal Genentics and Biology 2008. 45:17-27. Here the method of random insertion was exploited to create large numbers of mutants in A. niger and to screen the mutants having significant changes in laccase expression to identify new proteases or secretion-related genes that limiting production of heterologous protein.

As lower eukaryotic microorganisms, filamentous fungi are known for their robust ability to secrete large quantities of proteins. Such expression can reach as high as 40 g/L (Durand et al., Enzyme and Microbial Technology, 1988. 10(6):341-346) with a translational and post-translational modification process similar to that of mammalian cells except for their glycosylation. They are widely used in the chemical, pharmaceutical and food industries and it is generally regarded as safe (Schuster, E., et al., Appl Microbiol Biotechnol, 2002. 59(4-5):426-35). However, heterologous protein production in filamentous fungi is still relatively low as compared to the more common bacterial expression systems. Thus a need exists for more efficient expression systems that can produce heterologous proteins in greater quantities.

### SUMMARY

One aspect of the invention provides a filamentous fungal cell comprising at least one mutation, wherein the filamentous fungal cell has impaired ptrB activity and has altered expression of a protein of interest as compared to a corresponding parent filamentous fungal cell. In a preferred embodiment, the altered expression of the protein of interest is enhanced expression of the protein of interest.

Another aspect of the invention provides a filamentous fungal strain capable of expressing a heterologous protein, said strain comprising a mutation that results in decreased ptrB activity compared to a corresponding parent filamentous fungal strain. Yet another aspect of the invention provides methods for increasing expression of a protein of interest in a filamentous fungal host, said method comprising: (a) cultivating a mutant of a parent filamentous fungal cell under conditions conducive for production of the protein of interest, wherein the mutant comprises a first nucleic acid sequence encoding the protein of interest and a second nucleic acid sequence comprising a modification of at least one gene locus involved in the production of ptrB; and (b) isolating the protein of interest from the cultivation medium.

In certain embodiments, the mutated and parent filamentous fungi are protease deficient strains. In other embodiments, the filamentous fungi of the invention further comprise a mutation in, or flanking, a gene encoding a protease (in addition to the mutation resulting in impaired ptrB activity).

In certain embodiments, the mutation leading to impaired ptrB activity comprises a deletion in a noncoding region flanking the ptrB gene. In another embodiment, the mutation comprises an insertion mutation. Preferably, the insertion mutation is in a noncoding region flanking the ptrB gene. In certain embodiments, the insertion mutation comprises insertion of a selectable marker.

### BRIEF DESCRIPTION OF THE FIGURES

FIG. 1 provides an autoradiograph of a Southern analysis of 8 up-mutants strains. The linear pMW1 plasmid digested with HindIII restriction enzyme was used as the positive control (+) and the 800 by fragment of the hph gene was used as the probe. The genomic DNA was digested with HindIII, which cuts only once in plasmid. The arrow indicates the position of 4.3 kb full length plasmid DNA.

FIG. 2 provides an autoradiograph of a Southern analysis of the strain 16H2. Two bands were detected indicating that pMW1 integration occurred at only one locus. Lane M is the DNA marker of λ DNA digested by HindIII visualized under UV light and lane 16H2 represent the genomic DNA fragments hybridized to radiolabeled hph DNA probe.

FIG. 3 illustrates the integration of multiple copies of pMW1. The figure provides agarose gel analysis of PCR products. Using double restriction enzymes (BamHI and SmaI) digested genomic DNA as DNA template, and the specific primers dsp3, dsp4 and dsp5 and the random primer K7 respectively, the gradient products (indicated by the stars) were obtained, meaning that the SM-TAIL-PCR result was positive. Lane M is a 100 by DNA ladder.

FIG. 4 illustrates a comparison of mRNA level by Real-time RT-PCR method. Relative expression levels were determined after normalizing to the mRNA levels of strain GICC2773.

### DETAILED DESCRIPTION

The present invention relates recombinant filamentous fungal cells, such as Aspergillus cells, having impaired ptrB activity and capable of expressing at least one heterologous protein encoded by a heterologous gene. Nucleic acids and methods for making the mutant filamentous fungal cells are provided, as well as methods for using the cells for the altered production of heterologous proteins of interest.

All patents and publications, including all sequences disclosed within such patents and publications, referred to herein are expressly incorporated by reference. Unless defined otherwise herein, all technical and scientific terms used herein have the same meaning as commonly understood by one of ordinary skill in the art to which this invention belongs (See e.g., Singleton et al., DICTIONARY OF MICROBIOLOGY AND MOLECULAR BIOLOGY, 2D ED., © 1994, John Wiley and Sons, New York; and Hale and Marham, THE HARPER COLLINS DICTIONARY OF BIOLOGY, © 1991, Harper Perennial, NY, both of which provide one of skill with a general dictionary of many of the terms used herein). Any methods and materials similar or equivalent to the various embodiments described herein can be used in the practice or testing of the present invention.

It is intended that every maximum (or minimum) numerical limitation disclosed in this specification includes every lower (or higher) numerical limitation, as if such lower (or higher) numerical limitations were expressly written herein. Moreover, every numerical range disclosed in this specification is intended to include every narrower numerical range that falls within such broader numerical range, as if such narrower numerical ranges were all expressly written herein.

As used herein, the singular “a”, “an” and “the” includes the plural reference unless the context clearly dictates otherwise. Thus, for example, reference to a “host cell” includes a plurality of such host cells.

Unless otherwise indicated, nucleic acids are written left to right in 5′ to 3′ orientation; amino acid sequences are written left to right in amino to carboxyl orientation, respectively. The headings provided herein are not limitations of the various aspects or embodiments of the invention that can be had by reference to the specification as a whole. Accordingly, the terms defined immediately below are more fully defined by reference to the Specification as a whole.

As used herein, the term “impaired” or “impairment” refers to any method that decreases, but does not abolish, the functional expression of one or more genes or the functional activity of the resulting gene product (i.e. protein), fragments or homologues thereof, wherein the gene or gene product exerts its known function to a lesser extent than in the corresponding parent strain. It is intended to encompass any means of gene impairment include partial deletions, disruptions of the protein-coding sequence, insertions, additions, mutations, gene silencing (e.g. RNAi genes antisense) and the like.

As used herein, “deletion” of a gene refers to deletion of the entire coding sequence, deletion of part of the coding sequence, or deletion of the coding sequence including flanking regions.

As used herein “disruption” refers to a change in a nucleotide or amino acid sequence by the insertion of one or more nucleotides or amino acid residues, respectively, as compared to the parent or naturally occurring sequence. Accordingly, a “disruption sequence” or “disruption mutant” as used herein refers to a nucleic acid or amino acid sequence, typically a coding region sequence, that comprises an insertion of nucleotides or amino acids.

As used herein, “insertion” or “addition” in the context of a sequence refers to a change in a nucleic acid or amino acid sequence in which one or more nucleotides or amino acid residues have been added as compared to the endogenous chromosomal sequence or protein product.

As used herein, “non-revertable” refers to a strain which will naturally revert back to it corresponding parent strain with a frequency of less than 10⁻⁷.

As used herein, the term “corresponding parent strain” refers to the host strain from which a mutant is derived (e.g., the originating and/or wild-type strain).

As used herein, “strain viability” refers to reproductive viability. In some embodiments, the impairment of a gene does not deleteriously affect division and survival of the mutant under laboratory conditions.

As used herein “coding region” refers to the region of a gene that encodes the amino acid sequence of a protein.

As used herein “amino acid” refers to peptide or protein sequences or portions thereof. The terms “protein,” “peptide,” and “polypeptide” are used interchangeably.

As used herein, the term “heterologous protein” or “exogenous protein” refers to a protein or polypeptide that does not naturally occur in the host cell, and includes genetically engineered versions of naturally occurring endogenous proteins.

As used herein, “endogenous protein” or “native protein” refers to a protein or polypeptide naturally occurring in a cell.

As used herein, “host,” “host cell,” or “host strain” refer to a cell that can express a DNA sequence introduced into the cell. In some embodiments of the present invention, the host cells are Aspergillus sp.

As used herein, “filamentous fungal cell” refers to a cell of any of the species of microscopic fungi that grow as multicellular filamentous strands including but not limited to: Aspergillus sp., Rhizopus sp., Trichoderma sp., and Mucor sp.

As used herein, “Aspergillus” or “Aspergillus sp.” includes all species within the genus “Aspergillus,” as known to those of skill in the art, including but not limited to A. oryzae, A. niger, A. awamori, A. nidulans, A. sojae, A. japonicus, A. kawachi and A. aculeatus.

As used herein, “nucleic acid” refers to a nucleotide or polynucleotide sequence, and fragments or portions thereof, as well as to DNA, cDNA, and RNA of genomic or synthetic origin which may be double-stranded or single-stranded, whether representing the sense or antisense strand. It will be understood that as a result of the degeneracy of the genetic code, a multitude of nucleotide sequences may encode a given protein.

As used herein the term “gene” means a segment of DNA involved in producing a polypeptide and can include regions preceding and following the coding regions (e.g., promoter, terminator, 5′ untranslated (5′ UTR) or leader sequences and 3′ untranslated (3′ UTR) or trailer sequences, as well as intervening sequence (introns) between individual coding segments (exons).

As used herein, “homologous gene,” “gene homolog,” or “homolog” refers to a gene which has a homologous sequence and results in a protein having an identical or similar function. The term encompasses genes that are separated by speciation (La, the development of new species) (e.g., orthologous genes), as well as genes that have been separated by genetic duplication (e.g., paralogous genes).

As used herein, “homologous sequences” refers to a nucleic acid or polypeptide sequence having at least about 99%, at least about 98%, at least about 97%, at least about 96%, at least about 95%, at least about 94%, at least about 93%, at least about 92%, at least about 91%, at least about 90%, at least about 88%, at least about 85%, at least about 80%, at least about 75%, at least about 70% or at least about 60% sequence identity to a subject nucleotide or amino acid sequence when optimally aligned for comparison. In some embodiments, homologous sequences have between about 80% and 100% sequence identity, in some embodiments between about 90% and 100% sequence identity, and in some embodiments, between about 95% and 100% sequence identity.

Sequence homology can be determined using standard techniques known in the art (see e.g., Smith and Waterman, Adv. Appl. Math., 1981. 2:482; Needleman and Wunsch, J. Mol. Biol., 1970. 48:443; Pearson and Lipman, Proc. Natl. Acad. Sci. USA 1988. 85:2444; programs such as GAP, BESTFIT, FASTA, and TFASTA in the Wisconsin Genetics Software Package (Genetics Computer Group, Madison, Wis.); and Devereux et al., Nucl. Acid Res., 1984. 12:387-395).

Useful algorithms for determining sequence homology include: PILEUP and BLAST (Altschul et al., J. Mol. Biol., 1990. 215:403-410; and Karlin et al., Proc. Natl. Acad. ScL USA 1993. 90:5873-5787). PILEUP uses a simplification of the progressive alignment method of Feng and Doolittle (Feng and Doolittle, J. Mol. Evol.,1987. 35:351-360). The method is similar to that described by Higgins and Sharp (Higgins and Sharp, CABIOS 1989. 5:151-153). Useful PILEUP parameters including a default gap weight of 3.00, a default gap length weight of 0.10, and weighted end gaps.

A particularly useful BLAST program is the WU-BLAST-2 program (See, Altschul et al., Meth. Enzymol., 1996. 266:460-480). WU-BLAST-2 uses several search parameters, most of which are set to the default values. The adjustable parameters are set with the following values: overlap span=1, overlap fraction=0.125, word threshold (T)=11. The HSP S and HSP S2 parameters are dynamic values and are established by the program itself depending upon the composition of the particular sequence and composition of the particular database against which the sequence of interest is being searched. However, the values may be adjusted to increase sensitivity. A % amino acid sequence identity value is determined by the number of matching identical residues divided by the total number of residues of the “longer” sequence in the aligned region. The “longer” sequence is the one having the most actual residues in the aligned region (gaps introduced by WU-Blast-2 to maximize the alignment score are ignored).

As used herein, the term “vector” refers to any nucleic acid that can be replicated in cells and can carry new genes or DNA segments into cells. Thus, the term refers to a nucleic acid construct designed for transfer between different host cells. An “expression vector” refers to a vector that has the ability to incorporate and express heterologous DNA fragments (i.e., non-native DNA) in a cell. Many prokaryotic and eukaryotic expression vectors are commercially available. Selection of appropriate expression vectors is within the knowledge of those having skill in the art.

As used herein, the term “DNA construct” refers to a nucleic acid molecule generated recombinantly or synthetically, with a series of specified nucleic acid elements that permit transcription of a particular nucleic acid in a target cell (Le, vectors or vector elements, as described above). For example, DNA construct can be incorporated into a plasmid, chromosome, mitochondrial DNA, plastid DNA, virus, or nucleic acid fragment. In some embodiments, DNA constructs also include a series of specified nucleic acid elements that permit transcription of a particular nucleic acid in a target cell. In some embodiments, a DNA construct of the invention comprises a selectable marker.

Also as used herein, the term “DNA construct” (as well as “transforming DNA,” and “transforming sequence”) refers to DNA that is used to introduce sequences into a host cell or organism (i.e., “transform a host cell”). The DNA construct may be generated in vitro by PCR or any other suitable techniques. In some embodiments, the transforming DNA can include an incoming sequence, and/or can include an incoming sequence flanked by homology boxes. In yet a further embodiment, the transforming DNA comprises other non-homologous sequences, added to the ends (e.g., stuffer sequences or flanks). The ends can be closed such that the transforming DNA forms a closed circle (i.e., a plasmid), such as, for example, insertion into a vector.

As used herein, the term “plasmid” refers to a circular double-stranded (ds) DNA construct used as a cloning vector, and which forms an extrachromosomal self-replicating genetic element in many bacteria and some eukaryotes. In some embodiments, plasmids become incorporated into the genome of the host cell.

As used herein, the terms “isolated” and “purified” are used to refer to a molecule (e.g., a nucleic acid or polypeptide) or other component that is removed from at least one other component with which it is naturally associated.

As used herein, the term “altered expression” is construed to include an increase or decrease in production of a protein of interest by an altered (i.e., engineered) cell strain relative to the normal level of production from the corresponding unaltered parent strain (i.e., when grown under essentially the same conditions).

As used herein, the term “enhanced expression” is construed to include the increased production of a protein of interest by an altered (i.e., engineered) cell strain above the normal level of production from the corresponding unaltered parent strain (i.e., when grown under essentially the same conditions).

As used herein, the term “expression” refers to a process by which a polypeptide is produced. The process includes both transcription and translation of the gene. In some embodiments, the process also includes secretion of the polypeptide.

As used herein in the context of “introducing a nucleic acid sequence into a cell,” the term “introducing” (and in past tense, “introduced”) refers to any method suitable for transferring the nucleic acid sequence into the cell, including but not limited to transformation, electroporation, nuclear microinjection, transduction, transfection, (e.g., lipofection mediated and DEAE-Dextrin mediated transfection), incubation with calcium phosphate DNA precipitate, high velocity bombardment with DNA-coated microprojectiles, agrobacterium mediated transformation, and protoplast fusion.

As used herein, the terms “stably transformed” refers to a cell that has a non-native (heterologous) polynucleotide sequence integrated into its genome or as an episomal plasmid that is maintained for at least two generations.

As used herein “an incoming sequence” refers to a DNA sequence that is being introduced into a host cell. The incoming sequence can be part of a DNA construct, can encode one or more proteins of interest (e.g., heterologous protein), can be a functional or non-functional gene and/or a mutated or modified gene, and/or can be a selectable marker gene(s). For example, the incoming sequence can include a functional or sub-functional (e.g., impaired) version of a gene, preferably ptrB or fragment or a homolog thereof. In one embodiment, the incoming sequence includes two homology boxes.

As used herein, “homology box” refers to a nucleic acid sequence, which is homologous to the sequence of gene in the chromosome of a filamentous fungal cell. More specifically, a homology box is an upstream or downstream region having between about 80 and 100% sequence identity, between about 90 and 100% sequence identity, or between about 95 and 100% sequence identity with the immediate flanking coding region of a gene or part of a gene to be impaired according to the invention. These sequences direct where in the chromosome a DNA construct or incoming sequence is integrated and directs what part of the chromosome is replaced by the DNA construct or incoming sequence. While not meant to limit the invention, a homology box may include between about 1 base pair (bp) to 200 kilobases (kb). Typically, a homology box includes about between 1 by and 10.0 kb; between 1 by and 5.0 kb; between 1 by and 2.5 kb; between 1 by and 1.0 kb, and between 0.25 kb and 2.5 kb. A homology box may also include about 10.0 kb, 5.0 kb, 2.5 kb, 2.0 kb, 1.5 kb, 1.0 kb, 0.5 kb, 0.25 kb and 0.1 kb. In some embodiments, the 5′ and 3′ ends of a selective marker are flanked by a homology box wherein the homology box comprises nucleic acid sequences immediately flanking the coding region of the gene.

In an alternative embodiment, the transforming DNA sequence comprises homology boxes without the presence of an incoming sequence. In this embodiment, it is desired to delete the endogenous DNA sequence between the two homology boxes. Furthermore, in some embodiments, the transforming sequences are wild-type, while in other embodiments, they are mutant or modified sequences. In addition, in some embodiments, the transforming sequences are homologous, while in other embodiments, they are heterologous.

As used herein, the term “target sequence” refers to a DNA sequence in the host cell that encodes the sequence where it is desired for the incoming sequence to be inserted into the host cell genome. In some embodiments, the target sequence encodes a functional wild-type gene or operon, while in other embodiments the target sequence encodes a functional mutant gene or operon, or a non-functional gene or operon.

As used herein, a “flanking sequence” refers to any sequence that is either upstream or downstream of the sequence being discussed (e.g., for genes A-B-C, gene B is flanked by the A and C gene sequences). In some embodiments, the incoming sequence is flanked by a homology box on each side. In another embodiment, the incoming sequence and the homology boxes comprise a unit that is flanked by stuffer sequence on each side. In some embodiments, a flanking sequence is present on only a single side (either 3′ or 5′), and in other embodiments, it is on each side of the sequence being flanked. The sequence of each homology box is homologous to a sequence in the Aspergillus chromosome. These sequences direct where in the Aspergillus chromosome the new construct gets integrated and what part of the Aspergillus chromosome will be replaced by the incoming sequence. In some embodiments these sequences direct where in the Aspergillus chromosome the new construct gets integrated without any part of the chromosome being replaced by the incoming sequence. In some embodiments, the 5′ and 3′ ends of a selective marker are flanked by a polynucleotide sequence comprising a section of the desired chromosomal segment. In some embodiments, a flanking sequence is present on only a single side (either 3′ or 5′), and in other embodiments, it is present on each side of the sequence being flanked.

As used herein, the term “chromosomally integrated” refers to a sequence, typically a mutant gene (e.g., disrupted form of a native gene), that has become incorporated into the chromosomal DNA of a host cell. Typically, chromosomal integration occurs via the process of “homologous recombination,” wherein the homologous regions of the introduced (transforming) DNA align with homologous regions of the host chromosome. Subsequently, the sequence between the homologous regions is replaced by the incoming sequence in a double crossover. Thus, “chromosomally integrated” is used interchangeably herein with “homologously recombined” or “homologously integrated.”

As used herein, the terms “selectable marker” and “selective marker” refer to a nucleic acid capable of expression in host cell, which allows for ease of selection of those hosts containing the marker. Thus, the term “selectable marker” refers to genes that provide an indication that a host cell has taken up (e.g., has been successfully transformed with) an incoming nucleic acid of interest or some other reaction has occurred. Typically, selectable markers are genes that confer antimicrobial resistance or a metabolic advantage on the host cell to allow cells containing the exogenous DNA to be distinguished from cells that have not received any exogenous sequence during the transformation. Selective markers useful with the present invention include, but are not limited to, antimicrobial resistance markers (e.g., ampR; phleoR; specR; kanR; eryR; tetR; cmpR; hygroR and neoR; see e.g., Guerot-Fleury, Gene, 1995. 167:335-337; Palmeros et al., Gene 2000. 247:255-264; and Trieu-Cuot et al., Gene, 1983. 23:331-341), auxotrophic markers, such as trpC, pyrG and amdS, and detection markers, such as β-galactosidase.

As used herein, the term “promoter” refers to a nucleic acid sequence that functions to direct transcription of a downstream gene. In some embodiments, the promoter is appropriate to the host cell in which a desired gene is being expressed. The promoter, together with other transcriptional and translational regulatory nucleic acid sequences (also termed “control sequences”) is necessary to express a given gene. In general, the transcriptional and translational regulatory sequences include, but are not limited to, promoter sequences, ribosomal binding sites, transcriptional start and stop sequences, translational start and stop sequences, and enhancer or activator sequences.

A nucleic acid is “operably linked” when it is placed into a functional relationship with another nucleic acid sequence. For example, DNA encoding a secretory leader (i.e., a signal peptide), is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading phase. However, enhancers do not have to be contiguous. Linking is accomplished by ligation at convenient restriction sites. If such sites do not exist, the synthetic oligonucleotide adaptors or linkers are used in accordance with conventional practice.

As used herein, the term “hybridization” refers to the process by which a strand of nucleic acid joins with a complementary strand through base pairing, as known in the art.

A nucleic acid sequence is considered to be “selectively hybridizable” to a reference nucleic acid sequence if the two sequences specifically hybridize to one another under moderate to high stringency hybridization and wash conditions. Hybridization conditions are based on the melting temperature (Tm) of the nucleic acid binding complex or probe. For example, “maximum stringency” typically occurs at about Tm-5° C. (5° below the Tm of the probe); “high stringency” at about 5-10° C. below the Tm; “intermediate stringency” at about 10-20° C. below the Tm of the probe; and “low stringency” at about 20-25° C. below the Tm. Functionally, maximum stringency conditions may be used to identify sequences having strict identity or near-strict identity with the hybridization probe; while an intermediate or low stringency hybridization can be used to identify or detect polynucleotide sequence homologs.

Moderate and high stringency hybridization conditions are well known in the art. An example of high stringency conditions includes hybridization at about 42° C. in 50% formamide, 5×SSC, 5× Denhardt's solution, 0.5% SDS and 100 μg/ml denatured carrier DNA followed by washing two times in 2×SSC and 0.5% SDS at room temperature and two additional times in 0.1×SSC and 0.5% SDS at 42° C. An example of moderate stringent conditions include an overnight incubation at 37° C. in a solution comprising 20% formamide, 5×SSC (150 mM NaCl, 15 mM trisodium citrate), 50 mM sodium phosphate (pH 7.6), 5× Denhardt's solution, 10% dextran sulfate and 20 mg/ml denaturated sheared salmon sperm DNA, followed by washing the filters in 1×SSC at about 37-50° C. Those of skill in the art know how to adjust the temperature, ionic strength, etc. as necessary to accommodate factors such as probe length and the like.

As used herein, “recombinant” used in reference to a cell or vector refers to being modified by the introduction of a heterologous nucleic acid sequence, or a cell derived from a cell so modified. Thus, for example, recombinant cells express genes that are not found in identical form within the native (non-recombinant) form of the cell or express native genes that are otherwise abnormally expressed, underexpressed, overexpressed or not expressed at all as a result of deliberate human intervention. “Recombination, “recombining,” or generating a “recombined” nucleic acid is generally the assembly of two or more nucleic acid fragments wherein the assembly gives rise to a chimeric gene.

As used herein, the term “primer” refers to an oligonucleotide, whether occurring naturally as in a purified restriction digest or produced synthetically, which is capable of acting as a point of initiation of synthesis when placed under conditions in which synthesis of a primer extension product which is complementary to a nucleic acid strand is induced, (i.e., in the presence of nucleotides and an inducing agent such as DNA polymerase and at a suitable temperature and pH). Usually, the primer is single stranded for maximum efficiency in amplification. Most often, the primer is an oligodeoxyribonucleotide.

As used herein, the term “polymerase chain reaction” (“PCR”) refers to methods for amplifying DNA strands using a pair of primers, DNA polymerase, and repeated cycles of DNA polymerization, melting, and annealing (see, e.g., U.S. Pat. Nos. 4,683,195 4,683,202, and 4,965,188, which are hereby incorporated by reference herein).

As used herein, the terms “restriction endonucleases” and “restriction enzymes” refer to bacterial enzymes, each of which cut double-stranded DNA at or near a specific nucleotide sequence.

A “restriction site” refers to a nucleotide sequence recognized and cleaved by a given restriction endonuclease and is frequently the site for insertion of DNA fragments. In certain embodiments of the invention restriction sites are engineered into the selective marker and into 5′ and 3′ ends of the DNA construct.

The present invention provides filamentous fungi cells that are capable of producing a protein of interest at higher levels that the corresponding wild type filamentous fungal cells. In particular, the present invention relates to recombinant filamentous fungal microorganisms, such as Aspergillus species having decreased ptrB activity, resulting in altered expression of a protein of interest. In some embodiments, the decreased ptrB activity provides advantages such as improved production of a protein of interest.

In the present invention, the host cell is a filamentous fungal cell. Filamentous fungal cells useful with the present invention include, but are not limited to: Aspergillus sp., (e.g., A. oryzae, A. niger, A. awamori, A. nidulans, A. sojae, A. japonicus, A. kawachi and A. aculeatus); Rhizopus sp., Trichoderma sp. (e.g., Trichoderma reesei (previously classified as T longibrachiatum and currently also known as Hypocrea jecorina), Trichoderma viride, Trichoderma koningii, and Trichoderma harzianums)) and Mucor sp. (e.g., M. miehei and M. pusillus). In a preferred embodiment, the host cells are Aspergillus niger cells.

In some embodiments, the present invention may be used with particular strains of Aspergillus niger include ATCC 22342 (NRRL 3112), ATCC 44733, ATCC 14331, GICC2773 and strains derived therefrom. In some embodiments, the host cell is capable of expressing a heterologous gene. For example, the host cell may be a recombinant cell, which produces a heterologous protein. In other embodiments, the host is one that overexpresses a protein that has been introduced into the cell.

In some embodiments, the host strain is a mutant strain deficient in one or more genes such as genes corresponding to protease genes. For example, it is contemplated that an Aspergillus niger host cell may be used in which a gene encoding the major secreted aspartyl protease, such as aspergillopepsin has been deleted (see e.g., U.S. Pat. Nos. 5,840,570 and 6,509,171, which are hereby incorporated by reference herein).

In certain embodiments, the mutation leading to impaired ptrB activity comprises a deletion in a noncoding region flanking the ptrB gene. In another embodiment, the mutation comprises an insertion mutation. Preferably, the insertion mutation is in a noncoding region flanking the ptrB gene. In certain embodiments, the insertion mutation comprises insertion of a selectable marker. In some embodiments, wherein the genomic DNA is already known, the 5′ flanking fragment and the 3′ flanking fragment of the locus to be deleted is cloned by two PCR reactions, and in embodiments wherein the locus is disrupted or otherwise altered, the DNA fragment is cloned by one PCR reaction.

In some embodiments, the coding region flanking sequences include a range of about 1 bp to 2500 bp; about 1 bp to 1500 bp, about 1 by to 1000 bp, about 1 by to 500 bp, and 1 by to 250 bp. The number of nucleic acid sequences comprising the coding region flanking sequence may be different on each end of the gene coding sequence. For example, in some embodiments, the 5′ end of the coding sequence includes less than 25 by and the 3′ end of the coding sequence includes more than 100 bp.

In some embodiments, the incoming sequence comprises is a disruption sequence that comprises a selective marker flanked on the 5′ and 3′ ends with a fragment of the gene sequence. In other embodiments, when the DNA construct comprising the selective marker and gene, gene fragment or homologous sequence thereto is transformed into a host cell, the location of the selective marker renders the gene non-functional for its intended purpose. In some embodiments, the incoming sequence comprises the selective marker located in the promoter region of the gene. In other embodiments, the incoming sequence comprises the selective marker located after the promoter region of gene.

In yet other embodiments, the incoming sequence is a disruption sequence comprising the selective marker located in the coding region of the gene. In further embodiments, the incoming sequence comprises a selective marker flanked by a homology box on both ends. In still further embodiments, the incoming sequence includes a sequence that interrupts the transcription and/or translation of the coding sequence. In yet additional embodiments, the DNA construct includes restriction sites engineered at the upstream and downstream ends of the construct.

In one embodiment, the A. nidulans amdS gene provides a selectable marker system for the transformation of filamentous fungi useful with the present invention. The amdS gene codes for an acetamidase enzyme deficient in strains of Aspergillus and provides positive selective pressure for transformants grown on acetamide media. The amdS gene can be used as a selectable marker even in fungi known to contain an endogenous amdS gene or homolog, e.g., in A. nidulans (Tilburn et al. Gene 1983. 26: 205-221) and A. oryzae (Gomi et al. Gene 1991. 108:91-98). Background amdS activity of non-transformants can be suppressed by the inclusion of CsCI in the selection medium.

Methods for using amdS marker system in the transformation of industrially important filamentous fungi are established in the art (e.g., in Aspergillus niger (see e.g., Kelly and Hynes EMBO J. 1985. 4:475-479; Wang et al., Fungal Genet. Biol. 2008. 45(1):17-27); in Penicillium chrysogenum (see e.g., Beri and Turner, Curr. Genet. 2987. 11:639-641); in Trichoderma reesei (see e.g., Pentilla et al. Gene 1987. 61:155-164); in Aspergillus oryzae (see e.g., Christensen et al., Bio/technology 1988. 6:1419-1422); in Trichoderma harzianum (see e.g., Pe'er et al., Soil Biol. Biochem. 1990. 23:1043-1046); and U.S. Pat. No. 6,548,285, each of which is hereby incorporated by reference herein).

The DNA constructs comprising an incoming sequence may be incorporated into a vector (e.g., in a plasmid), or used directly to transform the filamentous fungal cell, thereby resulting in a mutant. Typically, the DNA construct is stably transformed resulting in chromosomal integration of the impaired gene which is non-revertable. Methods for in vitro construction and insertion of DNA constructs into a suitable vector are well known in the art.

Deletion and/or insertion of sequences is generally accomplished by ligation at convenient restriction sites. If such sites do not exist, synthetic oligonucleotide linkers can be prepared and used in accordance with conventional practice. (See, Sambrook (1989) supra, and Bennett and Lasure, MORE GENE MANIPULATIONS IN FUNGI, Academic Press, San Diego (1991) pp 70-76.). Additionally, vectors can be constructed using known recombination techniques (e.g., Invitrogen Life Technologies, Gateway Technology). Examples of suitable expression and/or integration vectors that may be used in the practice of the invention are provided in Sambrook et al., (1989) supra, Ausubel (1987) supra, van den Hondel et al. (1991) in Bennett and Lasure (Eds.) MORE GENE MANIPULATIONS IN FUNGI, Academic Press pp. 396-428 and U.S. Pat. No. 5,874,276. Exemplary vectors useful with the present invention include pBS-T, pFB6, pBR322, pUC18, pUC100 and pENTR/D.

In some embodiments, at least one copy of a DNA construct is integrated into the host chromosome. In some embodiments, one or more DNA constructs of the invention are used to transform host cells. For example, one DNA construct may be used to impair the ptrB gene and another construct may be used to inactivate a protease gene. Of course, additional combinations are contemplated and provided by the present invention.

Impairment occurs via any suitable means, including deletions, substitutions (e.g., mutations), disruptions, insertions in the nucleic acid gene sequence, and/or gene silencing mechanisms, such as RNA interference (RNAi). In one embodiment, the expression product of an impaired gene is a truncated protein with a corresponding change in the biological activity of the protein. In some embodiments, the impairment results in an attenuation of biological activity of the gene. In some embodiments, remaining residual activity will be less than 25%, 20%, 15%, 10%, 5%, or 2% compared to the biological activity of the same or homologous gene in a corresponding parent strain.

In some embodiments, impairment is achieved by deletion and in other embodiments impairment is achieved by disruption of the protein-coding region of the gene. In some embodiments, the gene is altered by homologous recombination.

In the cases a deletion is used to impair a gene, typically the deletion is partial. In some embodiments, a deletion mutant comprises deletion of one or more genes that results in a stable and non-reverting deletion. Flanking regions of the coding sequence may include from about 1 bp to about 500 by at the 5′ and 3′ ends. The flanking region may be larger than 500 by but typically does not include other genes in the region which may be impaired or deleted according to the invention.

In some embodiments, the disruption sequence comprises an insertion of a selectable marker gene into or near the protein-coding region. Typically, this insertion is performed in vitro by reversely inserting a gene sequence into or near the coding region sequence of the gene to be impaired. Flanking regions of the coding sequence may include about 1 bp to about 500 by at the 5′ and 3′ ends. The flanking region may be larger than 500 bp, but will typically not include other genes in the region. The DNA construct aligns with the homologous sequence of the host chromosome and in a double crossover event the translation or transcription of the gene is disrupted. For example, the ptrB chromosomal gene is aligned with a plasmid comprising a selective marker and the gene, part of the gene coding sequence, or a region flanking the coding sequence. In some embodiments, the selective marker gene is located within the gene coding sequence or on a part of the plasmid separate from the gene. The vector is chromosomally integrated into the host, and the host's gene is thereafter impaired by the presence of the marker inserted in or near the coding sequence or flanking region.

While not meant to limit the methods used for impairment, in some embodiments ptrB and homologous sequences may be impaired by this method, particularly by insertion of a selectable marker in the flanking sequences.

In some embodiments, impairment of the gene is by insertion in a single crossover event with a plasmid as the vector. For example, the vector is integrated into the host cell chromosome and the gene is altered by the insertion of the vector in the protein-coding sequence of the gene or in the regulatory region of the gene.

In alternative embodiments, impairment results due to mutation of the gene. Methods of mutating genes are well known in the art and include but are not limited to site-directed mutation, generation of random mutations, and gapped-duplex approaches (See e.g., U.S. Pat. No. 4,760,025; Moring et al., Biotech. 1984. 2:646; and Kramer et al., Nucleic Acids Res., 1984. 12:9441).

In some embodiments a mutant encompassed by the invention will exhibit altered expression and translation (i.e., protein production) of one or more endogenous and/or heterologous proteins of interest in comparison to the expression and translation of the same protein(s) by the corresponding parent strain of filamentous fungus.

In some embodiments, the mutants of filamentous fungal cells encompassed by the invention will produce the endogenous and/or heterologous proteins of interest in an amount at least about 0% to about 200% (or more) greater than the production of the same protein(s) in the corresponding parent strain. Accordingly, in some embodiments, the production of the protein(s) of interest by the mutant is at least about 0% to 100% greater, and in some embodiments is at least about 10% to 60% greater, including embodiments wherein production at least about 10%, 15%, 20%, 25%, 30%, 35%, 40%, 45%, 50%, and 55% greater, than the production of the endogenous and/or heterologous protein(s) in the corresponding parent strain.

In some embodiments of the present invention, the protein of interest produced by the mutant of a filamentous fungal cell is an intracellularly produced protein (i.e., an intracellular, non-secreted polypeptide). In other embodiments, the protein of interest is a secreted polypeptide. In addition, the protein of interest may be a fusion or hybrid protein. In some embodiments, the mutant exhibits altered production of a plurality of proteins, some of which are intracellular and some of which are secreted.

Proteins of interest useful with the present invention include enzymes known in the art, including, but not limited to those chosen from amylolytic enzymes, proteolytic enzymes, cellulytic enzymes, oxidoreductase enzymes and plant cell-wall degrading enzymes. More particularly, these enzyme include, but are not limited to amylases, glucoamylases, proteases, xylanases, lipases, laccases, phenol oxidases, oxidases, cutinases, cellulases, hemicellulases, esterases, perioxidases, catalases, glucose oxidases, phytases, pectinases, glucosidases, isomerases, transferases, galactosidases and chitinases. In some embodiments, enzymes include but are not limited to amylases, glucoamylases, proteases, phenol oxidases, cellulases, hemicellulases, glucose oxidases and phytases. In some embodiments, the polypeptide of interest is a protease, cellulase, glucoamylase or amylase.

In some embodiments, the protein of interest is a secreted polypeptide, which is fused to a signal peptide (i.e., an amino-terminal extension on a protein to be secreted). Nearly all secreted proteins use an amino- terminal protein extension, which plays a role in the targeting to and translocation of precursor proteins across the membrane. This extension is proteolytically removed by a signal peptidase during or immediately following membrane transfer.

In some embodiments of the present invention, the polypeptide of interest is a protein such as a protease inhibitor, which inhibits the action of proteases. Protease inhibitors are known in the art, for example the protease inhibitors belonging to the family of serine proteases inhibitors which are known to inhibit trysin, cathepsinG, thrombin and tissue kallikrein. Among the protease inhibitors useful in the present invention are Bowman-Birk inhibitors and soybean trypsin inhibitors (See, Birk, Int. J. Pept. Protein Res. 1985. 25:113-131; Kennedy, Am. J. Clin. Neutr. 1998. 68:1406S-1412S and Billings et al., Proc. Natl. Acad. Sci.1992. 89:3120-3124).

In some embodiments of the present invention, the polypeptide of interest is chosen from hormones, antibodies, growth factors, receptors, cytokines, etc. Hormones encompassed by the present invention include but are not limited to, follicle-stimulating hormone, luteinizing hormone, corticotropin-releasing factor, somatostatin, gonadotropin hormone, vasopressin, oxytocin, erythropoietin, insulin and the like. Growth factors include, but are not limited to platelet-derived growth factor, insulin-like growth factors, epidermal growth factor, nerve growth factor, fibroblast growth factor, transforming growth factors, cytokines, such as interleukins (e.g., IL-1 through IL-13), interferons, colony stimulating factors, and the like. Antibodies include but are not limited to immunoglobulins obtained directly from any species from which it is desirable to produce antibodies. In addition, the present invention encompasses modified antibodies. Polyclonal and monoclonal antibodies are also encompassed by the present invention. In some embodiments, the antibodies or fragments thereof are chimeric or humanized antibodies, including but not limited to: anti-p185^{Her2}, HuID10-, trastuzumab, bevacizumab, palivizumab, infliximab, daclizumab, and rituximab.

In a further embodiment, the nucleic acid encoding the protein of interest will be operably linked to a suitable promoter, which shows transcriptional activity in a fungal host cell. The promoter may be derived from genes encoding proteins either endogenous or heterologous to the host cell. The promoter may be a truncated or hybrid promoter. Further the promoter may be an inducible promoter. Typically, the promoter is useful in a Trichoderma host or an Aspergillus host. Suitable nonlimiting examples of promoters include cbh1, cbh2, egl1, egl2, and xyn1. In one embodiment, the promoter is one that is native to the host cell. Other examples of useful promoters include promoters from the genes of A. awamori and A. niger glucoamylase genes (glaA) (Nunberg et al., Mol. Cell Biol. 1984. 4:2306-2315 and Boel et al., EMBO J. 1984. 3:1581-1585); Aspergillus oryzae TAKA amylase; Rhizomucor miehei aspartic proteinase; Aspergillus niger neutral alpha-amylase; Aspergillus niger acid stable alpha-amylase; Trichoderma reesei stp1 and the cellobiohydrolase 1 gene promoter (see e.g., EP 0 137 280 A1, which is hereby incorporated by reference herein) and mutant, truncated and hybrid promoters thereof.

In some embodiments, the polypeptide coding sequence is operably linked to a signal sequence which directs the encoded polypeptide into the cell's secretory pathway. The 5′ end of the coding sequence may naturally contain a signal sequence naturally linked in translation reading frame with the segment of the coding region which encodes the secreted polypeptide. The DNA encoding the signal sequence typically is the sequence which is naturally associated with the polypeptide to be expressed. Typically, the signal sequence is encoded by an Aspergillus niger alpha-amylase, Aspergillus niger neutral amylase or Aspergillus niger glucoamylase. In some embodiments, the signal sequence is the Trichoderma cdh1 signal sequence which is operably linked to a cdh1 promoter.

Introduction of a DNA construct or vector into a host cell includes techniques such as transformation; electroporation; nuclear microinjection; transduction; transfection, (e.g., lipofection mediated and DEAE-Dextrin mediated transfection); incubation with calcium phosphate DNA precipitate; high velocity bombardment with DNA-coated microprojectiles; agrobacterium mediated transformation and protoplast fusion. General transformation techniques are known in the art (see, e.g., Ausubel et al., (1987), supra, chapter 9; and Sambrook (1989) supra, Campbell et al., Curr. Genet. 1989. 16:53-56 and THE BIOTECHNOLOGY OF FILAMENTOUS FUNGI, © 1992, Chap. 6. Eds. Finkelstein and Ball, Butterworth and Heinenmann, each of which is hereby incorporated by reference herein).

Production of heterologous proteins in filamentous fungal cell expression systems are also known in the art. For example, the expression of heterologous proteins in Trichoderma is described in Harkki et al., Enzyme Microb. Technol. 1991. 13:227-233; Harkki et al., Bio Techno. 1989. 7:596-603; EP 244,234; EP 215,594; and Nevalainen et al., “The Molecular Biology of Trichoderma and its Application to the Expression of Both Homologous and Heterologous Genes”, in MOLECULAR INDUSTRIAL MYCOLOGY, © 1992, Eds. Leong and Berka, Marcel Dekker Inc., NY, pp. 129-148; and U.S. Pat. Nos. 6,022,725 and 6,268,328, each of which is hereby incorporated by reference herein.

The expression of heterologous proteins in Aspergillus sp. is described in Cao et al., Sci. 2000. 9:991-1001; and U.S. Pat. No. 6,509,171, each of which is hereby incorporated by reference herein.

Transformants of the present invention can be purified using known techniques.

The filamentous fungal cells may be grown in conventional culture medium. The culture media for transformed cells may be modified as appropriate for activating promoters and selecting transformants. The specific culture conditions, such as temperature, pH and the like will be apparent to those skilled in the art. Typical culture conditions for filamentous fungi useful with the present invention are well known and may be found in the scientific literature such as Sambrook, (1982) supra, and from the American Type Culture Collection. Additionally, fermentation procedures for production of heterologous proteins are known per se in the art. For example, proteins can be produced either by solid or submerged culture, including batch, fed-batch and continuous-flow processes. Fermentation temperature can vary somewhat, but for filamentous fungi such as Aspergillus niger the temperature generally will be within the range of about 20° C. to 40° C., typically in the range of about 28° C. to 37° C., depending on the strain of microorganism chosen. The pH range in the aqueous microbial ferment (fermentation admixture) should be in the exemplary range of about 2.0 to 8.0. With filamentous fungi, the pH normally is within the range of about 2.5 to 8.0; with Aspergillus niger the pH normally is within the range of about 4.0 to 6.0, and typically in the range of about 4.5 to 5.5. While the average retention time of the fermentation admixture in the fermentor can vary considerably, depending in part on the fermentation temperature and culture employed, generally it will be within the range of about 24 to 500 hours, typically about 24 to 400 hours. Any type of fermentor useful for culturing filamentous fungi may be employed in the present invention. One useful embodiment with the present invention is operation under 15L Biolafitte (Saint-Germain-en-Laye, France).

Various assays are known to those of ordinary skill in the art for detecting and measuring activity of intracellularly and extracellularly expressed polypeptides. Means for determining the levels of secretion of a protein of interest in a host cell and detecting expressed proteins include the use of immunoassays with either polyclonal or monoclonal antibodies specific for the protein. Examples include enzyme-linked immunosorbent assay (ELISA), radioimmunoassay (RIA), fluorescence immunoassay (FIA), and fluorescent activated cell sorting (FACS). However, other methods are known to those in the art and find use in assessing the protein of interest (See e.g., Hampton et al., SEROLOGICAL METHODS, A LABORATORY MANUAL, © 1990, APS Press, St. Paul, Minn.; and Maddox et al., J. Exp. Med., 1983. 158:1211, each of which is hereby incorporated by reference herein). In some embodiments, the expression and/or secretion of a protein of interest are enhanced in a mutant. In some embodiments the production of the protein of interest is at least 100%, at least 95%, at least 90%, at least 80%, at least 70%, at least 60%, at least 50%, at least 40%, at least 30%, at least 20%, at least 15%, at least 10%, at least 5% and at least 2% greater in the mutant as compared to the corresponding parent strain.

Once the desired protein is expressed and, optionally, secreted, the protein of interest may be recovered and further purified. The recovery and purification of the protein of interest from a fermentation broth can be done by procedures known in the art. The fermentation broth will generally contain cellular debris, including cells, various suspended solids and other biomass contaminants, as well as the desired protein product.

Suitable processes for such removal include conventional solid-liquid separation techniques such as, e.g., centrifugation, filtration, dialysis, microfiltration, rotary vacuum filtration, or other known processes, to produce a cell-free filtrate. Often, it may be useful to further concentrate the fermentation broth or the cell-free filtrate prior to crystallization using techniques such as ultrafiltration, evaporation or precipitation.

Precipitating the proteinaceous components of the supernatant or filtrate may be accomplished by means of a salt, followed by purification by a variety of chromatographic procedures, e.g., ion exchange chromatography, affinity chromatography or similar art recognized procedures. When the expressed desired polypeptide is secreted the polypeptide may be purified from the growth media. Typically, the expression host cells are removed from the media before purification of the polypeptide (e.g., by centrifugation).

When the expressed recombinant desired polypeptide is not secreted from the host cell, usually the host cell is disrupted and the polypeptide released into an aqueous “extract” which is the first stage of purification. Typically, the expression host cells are collected from the media before the cell disruption (e.g., by centrifugation).

The manner and method of carrying out the present invention may be more fully understood by those of skill in the art by reference to the following examples, which examples are not intended in any manner to limit the scope of the present invention or of the claims directed thereto.

### EXAMPLES

The following Examples are provided in order to demonstrate and further illustrate specific embodiments and aspects of the present invention and are not to be construed as limiting the scope thereof.

A. Isolation of Insertional Mutants

A. niger strain GICC2773 was transformed with a 4.3 kb plasmid pMW1 (Kück, et al. Appl. Microbiol. Biotechnol. 1989. 31:358-365) using a protoplast-PEG transformation procedure (Wernars, K., et al., Mol Gen Genet, 1986. 205(2):312-7). Strain GICC2773 is a derivative of an AP-4 Aspergillus niger strain (see Ward et al., Appli. Microbiol. Biotechnol 1993. 39:738-743). GICC2773 includes a disruption mutant of the pepA gene and an integrated plasmid expressing a heterologous enzyme, laccase (Icc1 of the Tramete versicolor laccase gene) under glucoamylase promoter and terminator control. The GICC2773 strain is described in greater detail by Valkonen et al., Appl. Environ. Microbiol. 69(12); 6979-6986 (2003), which is hereby incorporated by reference herein.

Total of 8040 hygromycin resistant transformants were isolated on GMP agar plates (2% glucose, 2% maltose extract, 0.1% peptone, 1% agar) containing 200 μg/m1 of hygromycin B. Genomic DNA from 13 randomly picked transformants was analyzed by PCR analysis using primers specific to the hph gene. All of the transformants tested contained the hph gene. The same PCR analysis on A. niger strain GICC2773 was negative. Subsequently, a series of tests on 300 transformants for hygromycin resistance stability, colony morphology, sporulation capability and temperature sensitivity (ts) were conducted. The hygromycin resistance was stable in more than 99% of the insertional mutants after three passages, while colony morphology and sporulation capability exhibited significant polymorphism. In addition, one complete ts mutant and 7 partial ts mutants at 40° C. were detected. These data reflects the randomness of pMW1 integration in A. niger strain GICC2773.

Each of the 8040 transformants was visually screened using an ABTS assay that monitored color development on 96-well microtiter plates containing 200 μl of GMP agar in each well supplemented with 200 μg/ml of hygromycin B, 0.2 mM of ABTS and 0.1 mM of CuSO₄. 2 μl liquid suspensions of spores were inoculated into each well. After incubation at 30° C. for 3 to 4 days, up-mutants were identified by observing how fast the blue color developed and how dark the color was.

Mutants producing blue color earlier or darker were selected to be grown in S₄Y₂ broth (4% starch, 2% yeast extract, 0.5% KH₂PO₄, 0.5% corn flour) in flasks for three days before the supernatant were measured for laccase activity. The procedure was repeated three times and 8 mutants with increased extracellular laccase activity were isolated. These 8 mutants, when cultured in flasks with modified Promosoy broth (2% glucose, 8% starch, 4% Tryptic soy broth, 7% citrate sodium, 1.5% (NH₄)₂50₄, 0.1% NaH₂PO₄.H₂O, 0.1% MgSO₄.7H₂O, 0.07% Tween80, trace element) produced 56-200% greater laccase activity as compared to the parental strain, GICC2773. Extracellular laccase activity was measured using cell-free supernatant. Enzymatic reaction mix included 30 μl of diluted cell-free supernatant, 70 μl of 12.5 mmol/L ABTS and 2.9 ml of 0.1 mol/L acetate acid buffer adjusted to pH4.6. After incubation at 37° C. for 30 minutes, OD₄₂₀ was measured.

B. Examination of pMW1 Integrations in the Up-Mutants by Southern Blot

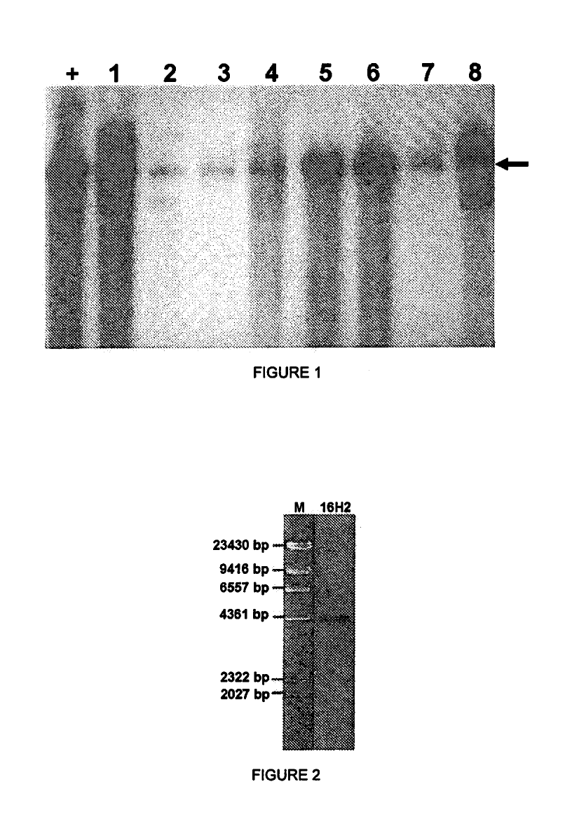

Genomic DNA was extracted from 8 laccase up-mutants using a benzyl chloride extraction according to Zhu, et al (Nucleic Acids Res. 1993. 21: 5279-5280). Extracted DNA was then digested with HindIII, an enzyme that cuts only once in pMW1. The probe was prepared by digestion of pMW1 with EcoRI, which generates an 800 by fragment containing half of the hph gene. This DNA fragment was then labeled using DIG High Primer DNA labeling and Detection starter Kit II (Roche Applied Science) for Southern blot analysis. This probe hybridized to a 4.3 kb fragment that was identical to the size of pMW1 in all eight laccase up-mutants, indicating a tandem repeat of several copies of pMW1 had integrated (FIG. 1). Interestingly, the Southern blot analysis reveled only two fragments in mutant strain 16H2. In addition to the 4.3 kb fragment, another fragment of 3.0 kb was hybridized to the probe, albeit with less intensity (FIG. 2). This pattern indicated that a tandem repeat of pMW1 was integrated into genome of strain 16H2 and it only involved one insertion site. Therefore, the increase of extracellular laccase expression (25% on average) in mutant strain 16H2 most likely results from host DNA disruption due to pMW1 integration. Strain 16H2 was chosen for detailed analysis of pMW1 integration.

C. Characterization of pMW1 Integration Locus in Mutant Strain 16H2 by SM-TAIL-PCR

It is known that transformants in A. niger typically contain multiple copies of plasmid inserted at a single locus resulting from random double strand break at the plasmid, followed by non-homologous end joining (NHEJ) (Walker, et al., Nature, 2001. 412:607-14). This unique arrangement interferes with specific binding of primers at the end copy, making it difficult to obtain a junction sequence employing typical PCR-based methods for identification of junction sequences, such as inverse PCR, linker-mediated PCR, and semi-random PCR. To overcome this problem, a modified method, termed SM-TAIL-PCR (Self-ligation Mediated TAIL-PCR), was developed to permit identification of the insertional site after plasmid insertion in the mutant strain 16H2. The unique features of SM-TAIL-PCR method include in two aspects. First, the genomic DNA was pre-digested twice at two closely positioned restriction sites flanking an antibiotic resistance gene on the integrating plasmid, thus preventing re-circularization of the tandem repeat. Preventing re-circularization eliminates the interference problem. Second, specific primers were designed to bind to sequence of the antibiotic resistance gene used for selecting transformants. The primers were very close to the above mentioned restriction sites, thus re-ligation of the digested template brings the unknown insertion site sequence next to the known specific priming sites. Keeping the junction in close proximity with the priming sites overcomes problems associated with not knowing the break site on the plasmid and producing very short products commonly found when using random non-specific primers of small oligos. This method provides an effective method for identification of the site of insertion, regardless of the multiplicity of plasmid copies at the insertion site.

The genomic DNA of mutant strain 16H2 was digested with 7 random combinations of two restriction enzymes based on multiple cloning sites (MCS)/polylinkers immediately flanking the hph cassette. The digested DNA was circularized to generate templates for TAIL-PCR. The upstream MCS/polylinker consists of HindIII, SalI and the downstream MCS/polylinker consists of BamHI, SmaI, KpnI and SacI. To prepare template for SM-TAIL-PCR, the mutant genomic DNA was digested with two restriction enzymes chosen either from the upstream polylinker (to isolate junction downstream of the hph gene) or the downstream polylinker (to isolate junction upstream of the hph gene). The digested DNA was then diluted and circularized to generate final templates for SM-TAIL-PCR. To amplify downstream junction of the hph gene, nested PCR was performed using a RAPD oligo composed of 10 nucleotides to pair with three primers usp1, usp2 and usp3, priming the minus strand of the hph gene at the 5′ end. Similarly, the junction upstream of the hph gene was amplified using nested primers dsp1, dsp2, dsp3, dsp4 and dsp5 (Table 1) that prime the plus strand of the hph gene at the 3′ end. The primers dsp3 to dsp5 were used for the tertiary PCR.

TABLE 1
Primer Sequences

| ID | sequences | SEQ ID |
|---|---|---|
| | | NO.: |
| P1 | 5′---CGAAATGAGCAGCGATCAGATTCG---3′ | SEQ ID |
| | | NO. 1 |
| P2 | 5′---GCACTTTGTACGGAGTACTGGGTTGTT---3′ | SEQ ID |
| | | NO. 2 |
| P3 | 5′---TTGATTCATTAAACATCATAATCCAAGCG---3′ | SEQ ID |
| | | NO. 3 |
| P4 | 5′---TCATTGACCCTTCCCTTACGATTCA---3′ | SEQ ID |
| | | NO. 4 |
| Pid | 5′---GTCCGAGGGCAAAGGAATAGAGTAGATG---3′ | SEQ ID |
| | | NO. 5 |
| Pout | 5′---CGAAATGAGCAGCGATCAGATTCG---3′ | SEQ ID |
| | | NO. 6 |
| usp1 | 5′---ATCAGGTCGGAGACGCTGTCGAACTT---3′ | SEQ ID |
| | | NO. 7 |
| usp2 | 5′---TCTCGACAGACGTCGCGGTGAGTTCA---3′ | SEQ ID |
| | | NO. 8 |
| usp3 | 5′---AAGCGATGAGGAACGCCGTTACATG---3′ | SEQ ID |
| | | NO. 9 |
| dsp1 | 5′---GGCGTATATGCTCCGCATTGGTCTT---3′ | SEQ ID |
| | | NO. 10 |
| dsp2 | 5′---TTGGTTGACGGCAATTTCGATGATG---3′ | SEQ ID |
| | | NO. 11 |
| dsp3 | 5′---GGCTGTGTAGAAGTACTCGCCGATAGTG---3′ | SEQ ID |
| | | NO. 12 |
| dsp4 | 5′---GTCCGAGGGCAAAGGAATAGAGTAGATG---3′ | SEQ ID |
| | | NO. 13 |
| dsp5 | 5′---GCTGGCGTAATAGCGAAGAG---3′ | SEQ ID |
| | | NO. 14 |
| P16H | 5′---GCCCAACGGACCTGAAAGTA---3′ | SEQ ID |
| | | NO. 15 |
| rp1 | 5′---GCACTTTGTACGGAGTACTGGGTTGTT---3′ | SEQ ID |
| | | NO. 16 |
| rp2 | 5′---CAGGCTTCCATTTCGTTGCT---3′ | SEQ ID |
| | | NO. 17 |
| K7 | 5′---AGCGAGCAAG---3′ | SEQ ID |
| | | NO. 18 |

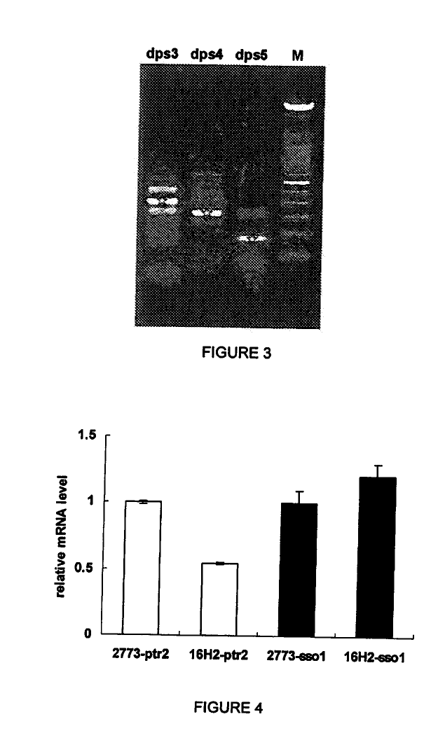

As an example, three primers were paired with a RAPD primer for three consecutive rounds of amplification (Nested PCR). As shown in FIG. 3, three PCR products with gradually decreased sizes were observed, indicating correct amplifications of targeted region, which was presumed to contain DNA near the pMW1 integration locus. 6 sets of such specific, correct PCR products were obtained from templates prepared with 7 different combinations of double digestions. The largest product was about 900 bp. The six PCR products were sequenced to identify non-pMW1 sequences and 6 primers were designed accordingly to pair with hph specific primers to amplify the junction DNA, using non-digested 16H2 genomic DNA as template. Amplification product was obtained from the primer P16H (Table 1) with an hph specific primer that targeted the minus strand at the 3′ end of the gene. Sequencing of the product indicated that the junction sequence flanked the pMW1 tandem repeat (data not shown). This sequence was compared to an A. niger sequence database (http://genome.jgi-psf.org/Aspni1/Aspni1.home.html) and to identify DNA sequences from both sides of the plasmid insertion.

D. Targeted Disruption of the Integration Locus 16H2

To further confirm that the identified integration locus affected laccase activity, targeted disruption of the same locus in A. niger strain GICC2773 was performed. An allele exchange plasmid pMW-16H2 (SEQ ID NO: 19) was constructed to carry an hph gene flanked by 1 kb of DNA homologous to the integration locus on each side. DNA flanking the insertion site was amplified use two sets of primers, P1/P2 (upstream of the insertion site) and P3/P4 (downstream of the insertion site). To create pMW1-16H2, the upstream DNA was inserted between the HindIII to SalI sites of pMW1 and the downstream DNA was inserted between the BamHI to SacI sites of pMW, thereby flanking the hph gene.

Plasmid pMW-16H2 was digested with Stul and Nael to obtain a 3.5 kb allele exchange cassette. After transformation, 43 hygromycin resistance transformants were isolated. PCR test using primers Pid and Pout identified one transformant of expected homologous recombination resulting insertion of the hph gene at the 16H2 locus. This transformant was named strain A16H2. Laccase activity in strains A16H2 and 16H2 was measured in the cell-free supernatant of cells cultured in shaking flasks. In addition, total soluble proteins was measured using Lowry assay (Lowry, O.H., et al., J Biol Chem, 1951. 193(1):265-75). The laccase activity in strain Δ16H2 was only 8% higher than that of strain GICC2773, a lower level than that of the strain 16H2. However, when the laccase activity was normalized to total soluble extracellular proteins, the increase in laccase activity was comparable between strains Δ16H2 and 16H2 (Table 2).

TABLE 2
Comparisons of the laccase activities of the strains Δ16H2, 16H2 and GICC2773

| | Laccase activity | | | | Total protein | | | Laccase activity/total protein | | | |
|---|---|---|---|---|---|---|---|---|---|---|---|
| | O.D₄₂₀ | | | | O.D₅₀₀ | | | O.D₄₂₀/O.D₅₀₀ | | | |
| strains | 1 | 2 | A* | %** | 1 | 2 | A* | 1 | 2 | A* | %** |
| Δ16H2 | 0.397 | 0.366 | 0.382 | 8.2 | 0.104 | 0.105 | 0.105 | 3.817 | 3.486 | 3.652 | 14.7 |
| 16H2 | 0.451 | 0.460 | 0.456 | 29.2 | 0.120 | 0.141 | 0.131 | 3.758 | 3.262 | 3.510 | 10.2 |
| GICC2773 | 0.359 | 0.347 | 0.353 | 0 | 0.108 | 0.114 | 0.111 | 3.324 | 3.044 | 3.184 | 0 |

A*: average value.
%**: percentage of the elevated level of laccase activity.

Southern blot analysis showed that the strain Δ16H2 contained additional copy of the hph cassette inserted at non-homologous region (data not shown). This illegitimate recombination might lead to a slower growth rate for the strain Δ16H2, hence lower laccase activity in the supernatant, but comparable when normalized using total soluble proteins. Nevertheless, our data confirmed that disruption of the integration locus 16H2 improved extracellular laccase expression.

E. Function of the Integration Locus in 16H2

Sequence analysis failed to identify any ORF within 1 kb of either side of integration locus 16H2. However, the search revealed an ptrB gene 2 kb upstream and an sso1 gene 1.2 kb downstream. To investigate which gene was affected by pMW1 insertion in the strain 16H2 that may lead to increased laccase expression, real-time RT-PCR was used to examine the transcription level of the ptrB and sso1 genes in strains GICC2773 and 16H2.

The quantitative reverse transcription-PCR (qRT-PCR) reactions were carried out in a 20 pl final volume containing: 13.8 μl of water, 1.6 μl of MgCl₂ (3 mM), 0.8 μl of each primer (rp1 and rp2; 10 mM), 2 μl of Fast Start DNA Master SYBR Green I and 1 μl RT product. The Real Time RT-PCR cycles were as follows: 10-min denaturation at 95° C., 40 cycles of amplification with 15 s of denaturation at 95° C., 5 s of annealing according to the melting temperatures of each pair of primers, 15 s of extension at 72° C. Fluorescence data collection was done at 76° C. Melting curve analyses were performed from 75 to 95° C. 1.5% agarose gel electrophoresis was used. Reaction containing no reverse transcripted total RNA samples was processed to demonstrate absence of genomic DNA contamination. The comparative threshold cycle (CT) method was used for the calculation of amplification folds (Tichopad, A., et al., Nucleic Acids Res, 2003. 31(20):e122; PfaffI, M. W., Nucleic Acids Res, 2001. 29(9):e45). The expression level of each gene was normalized by dividing it with the expression level of the 18S rRNA transcript. The expression level of the sso1 gene was not significantly affected, however, the expression level of the ptrB gene in the strain 16H2 was only half of that of the strain GICC2773 (FIG. 4).

This result suggests that the integration locus is part of a region regulating the expression of the ptrB gene. To test this hypothesis, a ptrB expression plasmid pGPT-ptrB was constructed by inserting the ptrB gene (see GenBank accession No. XM_{—}001395173) into pGPT vector (see M. Berka and C. Barnett, Biotech Adv, 1989 7(2):127-154, incorporated herein by reference). The ptrB sequence was inserted as a Bgl II restriction enzyme digested product of SEQ ID NO: 20. This ptrB expression plasmid contained the ptrB coding sequence under glaA promoter control and an additional more than 2 kb DNA. Strain 16H2 was co-transformed with plasmid pGPT-ptrB and p3SR2 (a plasmid containing the amdS gene of A. nidulans as a dominant selective marker; Hynes, et al., Mol Cell Biol, 1983. 3(8):1430-9) to test whether the level of laccase secretion could be restored to that of the wild type or even lower by the introduction of ptrB cassette. The protoplast preparation and transformation were performed according to protocols described in Wernars, K., et al., “Genetic analysis of Aspergillus nidulans AmdS+transformants.” Mol Gen Genet, 1986. 205(2):312-7. AmdS+ transformants were isolated and analyzed by PCR using primers rp1 and rp2 to screen for pGPT-ptrB transformants. 19 such transformants were identified. Extracellular laccase measurements showed reduced activity compared to that of the strain 16H2. 60% of transformants had laccase activity at the level of the strain GICC2773 or even lower (Table 3). Thus, improved laccase expression in the strain 16H2 likely is due to down regulation of the ptrB gene by pMW1 integration.

TABLE 3
Quantification of laccase activity

| Strain | OD₄₂₀ |
|---|---|
| GlCC2773 | 0.294 |
| 2 | 0.330 |
| 3 | 0.372 |
| 4 | 0.354 |
| 5 | 0.390 |
| 6 | 0.357 |
| 7 | 0.285 |
| 8 | 0.294 |
| 9 | 0.234 |
| 10 | 0.345 |
| 12 | 0.289 |
| 13 | 0.300 |
| 14 | 0.311 |
| 15 | 0.274 |
| 16 | 0.251 |
| 17 | 0.240 |
| 18 | 0.252 |
| 19 | 0.277 |
| 20 | 0.340 |
| 21 | 0.294 |
| 16H2 | 0.395 |

What is claimed is:

1. A filamentous fungal cell comprising at least one mutation, wherein the filamentous fungal cell has impaired prtB activity and has altered expression of a protein of interest as compared to a corresponding parent filamentous fungal cell.

2. The filamentous fungal cell of claim 1, wherein the filamentous fungal cell and corresponding parent filamentous fungal cell are protease deficient strains

3. The filamentous fungal cell of claim 1, wherein the mutation is located in a non-coding region adjacent to the ptrB gene locus.

4. The filamentous fungal cell of claim 3, wherein the mutation comprises a deletion in a noncoding region flanking the ptrB gene.

5. The filamentous fungal cell of claim 3, wherein the mutation comprises an insertion mutation.

6. The filamentous fungal cell of claim 5, wherein the insertion mutation comprises insertion of a selectable marker.

7. The filamentous fungal cell of claim 1, wherein the filamentous fungal cell is an Aspergillus species, a Rhizopus species, a Trichoderma species or a Mucor species.

8. The filamentous fungal cell of claim 1, wherein the filamentous fungal cell is an Aspergillus strain.

9. The filamentous fungal cell of claim 7, wherein the Aspergillus species is selected from the group consisting of A. oryzae, A. niger, A. awamori, A. nidulans, A. sojae, A. japonicus, A. kawachi and A. aculeatus. T

10. The filamentous fungal cell of claim 7, wherein the Trichoderma species is selected from the group consisting of Trichoderma reesei, Trichoderma viride, Trichoderma koningii, and Trichoderma harzianums.

11. The filamentous fungal cell of claim 1, wherein the altered expression of the protein of interest is enhanced expression of the protein of interest.

12. The filamentous fungal cell of claim 10, wherein the protein of interest is produced in an amount at least about 0% to 200% greater than the production of the same protein in the corresponding parent strain.

13. The filamentous fungal cell of claim 11, wherein the protein of interest is produced in an amount at least about 10% to 60% greater than the production of the same protein in the corresponding parent strain.

14. The filamentous fungal strain of claim 1, further comprising a mutation in, or flanking, a gene encoding a protease.

15. A filamentous fungal strain capable of expressing a heterologous protein, said strain comprising a mutation that results in decreased ptr2 activity compared to a corresponding parent filamentous fungal strain.

16. The filamentous fungal strain of claim 15, wherein the parent filamentous fungal cell is a protease deficient strain.

17. A method for increasing expression of a protein of interest in a filamentous fungal host, said method comprising:
cultivating a mutant of a parent filamentous fungal cell under conditions conducive for production of the protein of interest, wherein the mutant comprises a first nucleic acid sequence encoding the protein of interest and a second nucleic acid sequence comprising a modification of at least one gene locus involved in the production of ptr2; and
isolating the protein of interest.

18. The method of claim 17, wherein the parent filamentous fungal cell is a protease deficient strain.


SEQUENCE LISTING (20 sequences)

SEQ ID NO: 1 - DNA, 24 bp, Artificial Sequence
  Synthetic PCR primer
cgaaatgagcagcgatcagattcg

SEQ ID NO: 2 - DNA, 27 bp, Artificial Sequence
  Synthetic PCR primer
gcactttgtacggagtactgggttgtt

SEQ ID NO: 3 - DNA, 29 bp, Artificial Sequence
  Synthetic PCR primer
ttgattcattaaacatcataatccaagcg

SEQ ID NO: 4 - DNA, 25 bp, Artificial Sequence
  Synthetic PCR primer
tcattgacccttcccttacgattca

SEQ ID NO: 5 - DNA, 28 bp, Artificial Sequence
  Synthetic PCR primer
gtccgagggcaaaggaatagagtagatg

SEQ ID NO: 6 - DNA, 24 bp, Artificial Sequence
  Synthetic PCR primer
cgaaatgagcagcgatcagattcg

SEQ ID NO: 7 - DNA, 26 bp, Artificial Sequence
  Synthetic PCR primer
atcaggtcggagacgctgtcgaactt

SEQ ID NO: 8 - DNA, 26 bp, Artificial Sequence
  Synthetic PCR primer
tctcgacagacgtcgcggtgagttca

SEQ ID NO: 9 - DNA, 25 bp, Artificial Sequence
  Synthetic PCR primer
aagcgatgaggaacgccgttacatg

SEQ ID NO: 10 - DNA, 25 bp, Artificial Sequence
  Synthetic PCR primer
ggcgtatatgctccgcattggtctt

SEQ ID NO: 11 - DNA, 25 bp, Artificial Sequence
  Synthetic PCR primer
ttggttgacggcaatttcgatgatg

SEQ ID NO: 12 - DNA, 28 bp, Artificial Sequence
  Synthetic PCR primer
ggctgtgtagaagtactcgccgatagtg

SEQ ID NO: 13 - DNA, 28 bp, Artificial Sequence
  Synthetic PCR primer
gtccgagggcaaaggaatagagtagatg

SEQ ID NO: 14 - DNA, 20 bp, Artificial Sequence
  Synthetic PCR primer
gctggcgtaatagcgaagag

SEQ ID NO: 15 - DNA, 20 bp, Artificial Sequence
  Synthetic PCR primer
gcccaacggacctgaaagta

SEQ ID NO: 16 - DNA, 27 bp, Artificial Sequence
  Synthetic PCR primer
gcactttgtacggagtactgggttgtt

SEQ ID NO: 17 - DNA, 20 bp, Artificial Sequence
  Synthetic PCR primer
caggcttccatttcgttgct

SEQ ID NO: 18 - DNA, 10 bp, Artificial Sequence
  Synthetic PCR primer
agcgagcaag

SEQ ID NO: 19 - DNA, 7047 bp, Artificial Sequence
  Complete sequence of chemically synthesized plasmid pMW-16H2
gtcgactaccggtgagccgctcgacggggcgtcgagttgccgggccaatccctgagcttcgatagactgttccgggcctcatgggtggcggcgtctacatgcacatgcatgtaacggcgttcctcatcgcttggccccgccatgcagtcttcagggaccaaactccatcgccgctgctggaccgtatgtaacccccctcggcagtgtcacccgcaggagccggataatcgagaccttggtcaggccataaaggcgcgtcgtggggaagctcatatcgtatagcaacgggagacacgaggtaggtactcaagtacacatacacacacccagccgcccgtataaacagcttcaagaggggcgaatacttgaatatccctttggtcgctcttctgattttcgaggcttctccttccgccatcgtcactcacgcatatctcgtctttcacatcttacaccaggcaggacaaaccgtcaccatgagaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagagtagatgccgaccgggatccccttgattcattaaacatcataatccaagcgcaatcattaccacaaccatgcccaagcccacagacccaatgaacccaatccaaaccctacggtctagaatcctccccatccacagaacccaagtcccccaaaccatggaacccggacgagtaagggtccatagctaccacagctattctaccttgcacacacaacaatttgcagtcacaagtttgcactgaacccacttcacctaaccagacatccgaagattccccatacaaagtagatagtaagctaccgactgactgacttgcactctagaacccggctttccgttctatcccatttccttttccgttccgtgtgatgattgagtcattgcagcagcatgactgactaactacaaagaaataacctacaattctttcatttcttggactctttttgtgtggttatttcatgttactatacaggtaaaaattagccaccgttgtggatgattacctatgcttacaataaattcttgcttgcttgcttgctatgcacactacacaagtagaccagtcagtccatttagtgcatgaatttctggaggctggagaattacctaacttccattattcataacaatttgtccacgagactaaaaaactagcagataagcaagttccacttgtccgagtagtctaaattggtgtggtggggttgttcgagaataatttcaggcacgggtcggcttgggttgagtgggcactgcagggtctgctctgctctgcttgacggacggacgacctccgccggtgagtaatacttttgtttgatttcatacaagtaggatccaaccacaaaattcagccaggcaacgccataaaattatattgctatgctaatcatgatactaataacgagggtaaaaaaaaactatgcataatccgtacatcgtatgtacacccgccctttcacattctcatctgctctgcaggtgctcttacgcgggcctgcgcgacttcgcatatatcacgttcagcgcgtatacacgagaactgcgggtcaaaatattatagtacattagaaataatagagggtatcgagcggagtgagggcttttcagttctcgtgctccttcttctcctcataggccggcggggcaggagcatccggatcgtctgcgtcgagcgcattcagctggtcttcctggtcgttgaggtggtggtacaccagccagaagatgataccggcaacgaacgaagcgcaagccagacccacgaacatccacatgatggccgggtcataggcagcgggggtcagtgcctcggccagagcggaaccaaaggcgttggtgagcaggtacatggactgcacgaaggacttcatcgacggaggagccttggtgtaggcatactccaatccagacaccgacgcgaaaatctcagacagaccaatgaagacataggcaggcgtctggatagcaatgtgcacgtggttgccggtggtggacccatcaacctccgaagcagcacatccggggtgctcgtagcatggcccagcggagtaaatgaggtgctggacaatggcggcgtacatcatggccaaggaggcaacaacgaaacccagggaaatacgggtaatcgggcggaaacgaatgcgcaagcgacgcagcaggggatagacaagggtctccaggacggggatgaagacaatgatggagataggatcaaagttctgcatcaagtcgttcgggattccgtgaccctccatctggccggcctgggtgacaaagttgctggagaattgaccgtaaacgacccagtagatcgggtagaaacagaacacacggcaagcaaccagcgcacgcttgagctcgaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctttttgagtacaatatgttagctcaatttcccctgacgctgcttccccacttcacaatctccagcaacggcagaaccatgccgtcaagtttcacctcattgactctcatgttagcatgcatattaattccaacatttccatcttcattccaaacagctgcttaggccttcaacacaccaaccaaaggcataagcaacccagctcatctcatctcagctcagcatcttccacaatggaatttccacagcagctccacagccgcgatctagcttggccagcgtgtgcctgtctaacccacaagcgagggtaaaattagcaacaacgaactccattcccgatgcaagtgtgtgcagccattcttcgaatcatgcttctaggcaggaaatgtcggtgtctaaatggcaacgtgcccaccgccacactatatcagcgtagtttagcttgagctgagcctcaagtgggtgaaaaaccagacactgccccattccctcggtcggggcccgtgaataaacctcaggcagccgttttaccgctccgtcaagaggtcacatgctacgaggcaatgagctctcacgttactccatcttatccctcgcatattgtcgcgtggcccaaatgcttgatcgctttgaagaactacacaggtgggttcacgcgacgaaccttatatctaatgtatcgcagcaagggatgttgaacatatatctagtacctacatgaaacagacatatgccatattaaattaatggtctagatgaactactaatagtagtagcaaagtcgaatgttctcctactaaccctctacaaaatcaaccctctttcaatcgaagccaactgaacccttcaacaccagtcgaacataagagtagtacgagacataacacctcctatcatcctgagcgacagcccaacggacctgaaagtattaagtattacccaaagtacaaatgaaaccagaatacaaagcctcacagacacaaaacaataaatcatctactacccatccaacaacccagtactccgtacaaagtgctcgac

SEQ ID NO: 20 - DNA, 4024 bp, Artificial Sequence
  Chemically synthesized ptrB gene fragment
caaagatctatgatatcccagcccgcaacaacagggccacggcaccgaaactattgtgaacgaaaaggcggcattcaccgaggccagcgaaaaggccctcagtcccacccccgacggcgaagaacccaacgctgaggaagtcgctactcttcgccatgttgccgagcatctgcccatctccgcatggcttgtcgccgttgtcgagctgtgcgagcgtttcacctactacggaatgtctggcatgttccagaactacgttgatcgtccactggacggaagtgagggacgcggtgccctaggcatgggtcaccaaggagccactggtctcaccactttcttccagttctggtgttatggttcgttcactactgcataaaatgcttgggatcatggactgacatttgctcaaagttacccccatcatcagtgccatcgtcgctgatcagtacctcggtaaatacaagaccatcgtggttttctgtatcatttacttggtcggactcctcatcctggtttgcacgtccatcccgacggctcttcaccatggagccggtgttggtggcttcattgtgtctatcctgatcattggtctgggaactggcggtatcaagagtaacgttgctcctctgattgccgaccagtacaagcgcaagaagatggccatgtccaccaccaagaagggcgagcgtgttgtcattgacccttcccttacgattcagcgcatttacatgatcttctacggctgtatcaacgtgggatcgctgtctctgttggccactccttatatggagaagtacattggcttctggtccggttatctgctttgcctgtgcatgttcgctgtcggtaccatggtgctcatcttcggccgcaagtactacgtcgttcgccctccccagggctccattatcaccgacgccttcaaggcgctgtggatcatgattgtcaaccgcaacatggatgctcccaagcccagctggcaggcagccaacaacggtaccaggaccaacgtgccctgggacgaccacttcatcgacgagctcaagcgtgcgctggttgcttgccgtgtgttctgtttctacccgatctactgggtcgtttacggtcaattctccagcaactttgtcacccaggccggccagatggagggtcacggaatcccgaacgacttgatgcagaactttgatcctatctccatcattgtcttcatccccgtcctggagacccttgtctatcccctgctgcgtcgcttgcgcattcgtttccgcccgattacccgtatttccctgggtttcgttgttgcctccttggccatgatgtacgccgccattgtccagcacctcatttactccgctgggccatgctacgagcaccccggatgtgctgcttcggaggttgatgggtccaccaccggcaaccacgtgcacattgctatccagacgcctgcctatgtcttcattggtctgtctgagattttcgcgtcggtgtctggattggagtatgcctacaccaaggctcctccgtcgatgaagtccttcgtgcagtccatgtacctgctcaccaacgcctttggttccgctctggccgaggcactgacccccgctgcctatgacccggccatcatgtggatgttcgtgggtctggcttgcgcttcgttcgttgccggtatcatcttctggctggtgtaccaccacctcaacgaccaggaagaccagctgaatgcgctcgacgcagacgatccggatgctcctgccccgccggcctatgaggagaagaaggagcacgagaactgaaaagccctcactccgctcgataccctctattatttctaatgtactataatattttgacccgcagttctcgtgtatacgcgctgaacgtgatatatgcgaagtcgcgcaggcccgcgtaagagcacctgcagagcagatgagaatgtgaaagggcgggtgtacatacgatgtacggattatgcatagtttttttttaccctcgttattagtatcatgattagcatagcaatataattttatggcgttgcctggctgaattttgtggttggatcctacttgtatgaaatcaaacaaaagtattactcaccggcggaggtcgtccgtccgtcaagcagagcagagcagaccctgcagtgcccactcaacccaagccgacccgtgcctgaaattattctcgaacaacccaccacaccaatttagactactcggacaagtggaacttgcttatctgctagttttttagtctcgtggacaaattgttatgaataatggaagttaggtaattctccagcctccagaaattcatgcactaaatggactgactggtctacttgtgtagtgtgcatagcaagcaagcaagcaagaatttattgtaagcataggtaatcatccacaacggtggctaatttttacctgtatagtaacatgaaataaccacacaaaaagagtccaagaaatgaaagaattgtaggttatttctttgtagttagtcagtcatgctgctgcaatgactcaatcatcacacggaacggaaaaggaaatgggatagaacggaaagccgggttctagagtgcaagtcagtcagtcggtagcttactatctactttgtatggggaatcttcggatgtctggttaggtgaagtgggttcagtgcaaacttgtgactgcaaattgttgtgtgtgcaaggtagaatagctgtggtagctatggacccttactcgtccgggttccatggtttgggggacttgggttctgtggatggggaggattctagaccgtagggtttggattgggttcattgggtctgtgggcttgggcatggttgtggtaatgattgcgcttggattatgatgtttaatgaatcaagcactttgtacggagtactgggttgttggatgggtagtagatgatttattgttttgtgtctgtgaggctttgtattctggtttcatttgtactttgggtaatacttaatactttcaggtccgttgggctgtcgctcaggatgataggaggtgttatgtctcgtactactcttatgttcgactggtgttgaagggttcagttggcttcgattgaaagagggttgattttgtagagggttagtaggagaacattcgactttgctactactattagtagttcatctagaccattaatttaatatggcatatgtctgtttcatgtaggtactagatatatgttcaacatcccttgctgcgatacattagatataaggttcgtcgcgtgaacccacctgtgtagttcttcaaagcgatcaagcatttgggccacgcgacaatatgcgagggataagatggagtaacgtgagagctcattgcctcgtagcatgtgacctcttgacggagcggtaaaacggctgcctgaggtttattcacgggccccgaccgagggaatggggcagtgtctggtttttcacccacttgaggctcagctcaagctaaactacgctgatatagtgtggcggtgggcacgttgccatttagacaccgacatttcctgcctagaagcatgattcgaagaatggctgcacacacttgcatcgggaatggagttcgttgttgctaattttaccctcgcttgtgggttagacaggcacacgctggccaagctagatcgcggctgtggagctgctgtggaaattccattgtggaagatgctgagctgagatgagatgagctgggttgcttatgcctttggttggtgtgttgaaggcctaagcagctgtttggaatgaagatggaaatgttggaattaatatgcatgctaacatgagagtcaatgaggtgaaacttgacggcatggttctgccgttgctggagattgtgaagtggggaagcagcgtcaggggaaattgagctaacatattgtactcaaaaagctttattacacttaatttcatcgcgaatctgatcgctgctcatttcgttaacagtgtcggggacgtgtatggggtagattgggcgcgtagtcatggaaagctgggtgg